(19) United States
(12) Patent Application Publication
(10) Pub. No.: US 2002/0081322 A1
(43) Pub. Date: Jun. 27, 2002

(54) GEL-TYPE OIL FREE COSMETIC

(76) Inventors: CLEMENT LAWSON, BAY SHORE, NY (US); ISAAC D. COHEN, BROOKLYN, NY (US)

Correspondence Address: KAREN A. LOWNEY, ESQ, ESTEE LAUDER COMPANIES, 125 PINELAWN ROAD, MELVILLE, NY, 11747, US

(21) Appl. No.: 09/441,832

(22) Filed: Nov. 17, 1999

(51) Int. Cl. A61K 7/42; A61K 7/021; A61K 7/025; A61K 7/035; A61K 7/06; A61K 7/11; A61K 7/075; A61K 7/08; A61K 6/00; A61K 7/00

(52) U.S. Cl. 424/401; 424/59; 424/63; 424/64; 424/69; 424/70.1; 424/70.12; 424/70.27; 514/845; 514/846; 514/847; 514/944; 514/945

(57) ABSTRACT

The invention relates to non-emulsion, topical gel compositions which comprise an aliphatic polyhydric alcohol, an alkali salt of a fatty acid ester, a carbohydrate-based or cholesterol-based surfactant/emulsifier, or combination thereof, water, and substantially no oil.

### FIELD OF THE INVENTION

The invention relates to cosmetic compositions. More specifically, the invention relates to oil-free cosmetic compositions with excellent moisturizing properties.

### BACKGROUND OF THE INVENTION

Color cosmetics come in many forms. Probably the most common of these, especially for application of color to the face, is a liquid or cream foundation that the user applies to the skin with her fingertips or a sponge. Typically, these products are water and oil emulsions, containing a significant proportion of an oil component. While acceptable, or even desirable, for a large segment of users, the presence of oil makes it less desirable for those users who suffer with oily skin. In addition, the oil can feel heavy and unpleasant in warm weather. The same is true of a large number of skin care products, which are also frequently emulsions, and because of the presence of oil, may not be suitable for use with all skin types.

There are also stick-based formulations that provide an alternate approach to application of color. The most familiar of these are the lipstick formulation which rely largely on wax to solidify an oil-based formulation; such sticks can also be used in formulating foundations, but because they are primarily composed of waxes and oils, they can be less than ideal for oily skin, and can also leave a heavy, greasy feeling. Examples of these are found in U.S. Pat. No. 5,538,718; U.S. Pat. No. 4,725,432; U.S. Pat. No. 5,466,457; JP 89033442; and JP 89041602.

There thus continues to be a need for color cosmetics, particularly foundations, as well as skin-care products, that are not primarily oil-based, and which provide for ease of application and comfortable feel on the skin. The present invention now provides such compositions.

### SUMMARY OF THE INVENTION

The present invention provides a non-emulsion, semi-solid or soft cosmetic composition comprising an aliphatic polyhydric alcohol, an alkali salt of a fatty acid ester, a carbohydrate-based surfactant/emulsifier, water, and substantially no oil. The compositions of the invention provide a moisturizing, non-oily base for color cosmetics or for skin treatment products.

### DETAILED DESCRIPTION OF THE INVENTION

The compositions of the present invention are based in part on the “soap stick” technology that is so commonly used in the manufacture of deodorant or antiperspirant sticks. Sticks of this type are described, for example, in U.S. Pat. Nos. 5,462,736; 5,407,668; 4,268,498; 4,759,924; 5,120,541; 5,128,123; 4,252,789; 4,725,430; 2,890,987; and EP 450597. Unlike deodorant sticks, however, the cosmetics of the invention are not solid, but rather with a soft gel- or mousse-like texture. This use of a soap-gelled composition as the basis of a non-stick or soft cosmetic is a novel approach to the use of this type of technology, and provides a non-oily, easy-to-use makeup or skin care product having not only a smooth, elegant texture, but also unexpected moisturization and cooling properties.

The base of the composition is a polyhydric alcohol which is gelled by a soap. For the present purposes, the appropriate soaps used as gelling agents are low molecular weight amine and alkali metal salts, such as sodium and potassium, of fatty acids. In a preferred embodiment, the soap gelling agent is selected from sodium, potassium or amine salt of a C12-C22 fatty acid. Such gelling agents are known in the art, and are described, for example, in U.S. Pat. No. 5,128,123, the contents of which are incorporated herein by reference. In a particularly preferred embodiment, the gelling agent is a salt of behenic acid, and most preferably is sodium behenate. The latter is gentler than the more commonly used sodium stearate, and is thus well adapted for the contemplated use on skin, especially facial skin. The gelling agent is preferably present in an amount adequate to achieve a gel structure, but inadequate to product the hardness of a stick. Typically the amount used will be from about 1 to about 5%, more preferably from about 2 to about 4%. It will be understood also that the soap may be added directly to the composition, or may be formed in situ by addition of the component fatty acid and alkali metal hydroxide individually.

In order to prepare the final composition, the gelling agent must be solubilized. To achieve solubilization of the soap, at least one polyhydric alcohol is used. Strictly speaking, a large variety of polyhydric alcohols are capable of being used as solvents for the gelling agent; it is preferred that the polyhydric alcohol be a C2-C6 alcohol, containing from 2-6 hydroxyl groups. Preferably, the alcohol solvent is present in an amount of from about 5% to about 50% of the composition, more preferably, from about 10% to about 40%. Preferred is isoprene glycol, or any combination of isoprene glycol with another polyhydric alcohol, for example a combination of isoprene glycol and butylene glycol.

To achieve a soft gel or mousselike texture, rather than the solid stick form usually associated with soap-gelled products, the compositions of the invention also will comprise a carbohydrate-based or sterol-based surfactant or emulsifier. The carbohydrate surfactant may be of the oil-in-water type, or the water-in-oil type, and the carbohydrate upon which it is based is preferably an mono- or disaccharide, such as sucrose or glucose. Particularly preferred are fatty acid esters of sucrose and glucose, or ethers with ethylene or propylene glycol, with or without fatty acids. Examples of such surfactants include methyl glucose dioleate, PEG-120 methyl glucose dioleate, PPG-20 methyl glucose ether, PPG-20 methyl glucose ether distearate, PPG-20 methyl glucose distearate, PEG-20 methyl glucose distearate, methyl gluceth-10, methyl glucoside dioleate, sucrose stearate, sucrose distearate, and the like. Useful sterol-based surfactant/emulsifiers include cholesterol and phytosterols, or derivatives thereof. The surfactant is employed in an amount of about 1 to about 10% by weight, preferably about 2 to about 6%.

The remainder of the composition can be made up primarily with water, which will typically be from about 10-60%, preferably about 20 to about 40% by weight of the total composition. The water can be added directly to the composition, or may be provided in the form of an aqueous liposome suspension encapsulating water. Particularly preferred are non-phospholipid-based liposomes, such as are described in, for example, 4,895,452, 4,855,090, or 4,911,928. The composition of the invention contains substantially no oil, by which is meant there is less than about 5%, preferably less than about 3%, so as to avoid the creation of an emulsion, which may alter the desirable mousse texture.

The composition may also contain at least one pigment component. By “pigment” is meant any cosmetically acceptable colorant, such as are commonly used in color cosmetics. The pigments can be either inorganic or organic. Examples of useful inorganic pigments include iron oxides (yellow, red, brown or black), ferric ammonium ferrocyanide(blue), manganese violet, ultramarine blue, chromium oxide(green), talc, kaolin, titanium dioxide(white), zinc oxide, and mixtures thereof. Other useful pigments are pearlants such as mica, bismuth oxychloride and treated micas, such as titanated micas.

The organic pigments include natural colorants and synthetic monomeric and polymeric colorants. Exemplary are phthalocyanine blue and green pigment, diarylide yellow and orange pigments, and azo-type red and yellow pigments such as toluidine red, litho red, naphthol red and brown pigments. Also useful are lakes, which are pigments formed by the precipitation and absorption of organic dyes on an insoluble base, such as alumina, barium, or calcium hydrates. Particularly preferred lakes are primary FD&C or D&C lakes and blends thereof.

Also included are copolymer pigments that are water insoluble, e.g., nylon powder, polyethylene, and polyesters. The polyesters can include linear, thermoplastic, crystalline or amorphous materials produced using one or more diols and one or more dicarboxylic acids copolymerized with colorants. Other pigments to be used in the invention will be apparent to one of ordinary skill in the art. The amount of pigment used will vary depending on the nature of the final product, but will generally be in the range of from about 0.1 to about 40% by weight of the total composition.

Particularly preferred pigments are water-dispersible pigment, i.e., a pigment that is readily dispersed in an aqueous vehicle at room temperature without the necessity for high levels of surfactants. Such pigments are coated with a non-ionic coating, i.e., an uncharged material, so as to render them water-dispersible. In a preferred embodiment, the coating is a water-dispersible polymer, more preferably a water dispersible silicone polymer, most preferably, dimethicone copolyol. Such coated pigments are available under the name Chroma-philic, made by Enhance Technologies, Tonowanda, N.Y. Other water-dispersible pigments, coated with the polysaccharide galactoarabinan, are available from Whittaker, Clark & Daniels, South Plainfield, N.J.

The mousse may also optionally contain one or more other components which assist in enhancing the look and feel of the product on the skin. An optional component of the formulation are spherical powders which can aid in softening the appearance on the skin. Such materials are known in the cosmetic industry for their light-scattering properties on the skin. Powders of this type may include, but are not limited to, powders comprising(with examples of commercially available sources) calcium aluminum borosilicate (Luxsil™), PMMA (Microsphere M-100), polyethylene (polyethylene Cl 2080), methyl methacrylate crosspolymer (Covabeads LH85), nylon-12 (Orgasol 2002 O Nat Cos C, or Kobo, SP10 and SP 500), polymethyl silsesquioxane (Kobo, Tospearl 145, Tospearl 2000), polypropylene (Kobo, PP), spherical silicas (Kobo, MSS-500), polyurethane (Kobo, BPD-500) or ethylene/acrylic acid copolymer (Flobeads EA209). These powders, when used, are present in an amount of from about 0.001% to about 20%, preferably about 1% to about 10%, by weight of the total composition. Other non-spherical cosmetic powders, for example, boron nitride, mica, kaolin, magnesium carbonate, polyethylene powder, methylacrylate powder, silica, calcium carbonate, magnesium silicate, and the like, can also be employed to enhance feel, ease of application, and light scattering on the skin.

The mousses of the invention are useful as makeup products, including, but not limited to, foundation, blushes, eyeshadows, eyeliners, lipglosses, and concealers. The mousses may also be used as the base for any type of skin care products, such as moisturizers, sunscreens, self-tanners, and the like. Because of the high levels of water in the mousse, the compositions can also contain one or more water-soluble actives, as either a makeup or skin care product. Examples of water-soluble actives that may be included are water soluble preservatives and antioxidants; water soluble actives or skin conditioning agents, for example, humectants, such as hyaluronic acid salts, hydrogels, or glycerol or elastin; collagen; alpha- and beta-hydroxy acids; water-soluble vitamins; or milk protein. The composition can also include therapeutic water-soluble actives for topical application such as those that improve or eradicate age spots, keratoses and wrinkles, analgesics, anesthetics, anti-acne agents, antibacterials, antiyeast agents, antifungal agents, antiviral agents, antidandruff agents, antidermatitis agents, antipruritic agents, antiemetics, antimotion sickness agents, anti-inflammatory agents, antihyperkeratolytic agents, anti-dry skin agents, antiperspirants, antipsoriatic agents, antiseborrheic agents, hair conditioners and hair treatment agents, antiaging agents, antiwrinkle agents, antiasthmatic agents and bronchodilators, sunscreen agents, antihistamine agents, skin lightening agents, depigmenting agents, wound-healing agents, vitamins, corticosteroids, tanning agents, sunscreens or hormones.

The mousse cosmetics of the invention provide a unique combination of features. The mousse form makes the product very elegant in feel and appearance, and it is lightweight on the skin. Unlike other soap-gelled products, it is not a hard solid stick, but rather a semi-solid gel. To show the difference in hardness of the compositions of the invention, penetrometer readings were conducted on a variety of different soap-gelled product types. Using a penetrometer cone having a diameter of 1.25 inches, and a weight of 22.5 g, for a period of 15 seconds, a typical solid antiperspirant stick gives a penetrometer reading of about 2.9-4.0 mm; in contrast, products of the invention show readings ranging from about 10 mm(a firm gel) to about 13 mm (a semi-firm or mousse-like gel), demonstrating the significant differences in the aesthetics of the present products relative to solid sticks.

In addition, the absence of any type of oil or oily substance in the product distinguishes it from most color cosmetics, and thus makes it ideal for the large segment of the makeup-wearing population who experiences problems with oily skin. Finally, the large amount of water in the composition provides the benefit of adding water directly to the skin, as well as a burst of cool moisture on application, without the drippiness that is frequently associated with products having a high water content.

### EXAMPLES

I. This example illustrates a gel-makeup composition of the present invention:

| Ingredient | Weight % |
|---|---|
| Phase I | |
| Black iron oxide | 0.10 |
| Red iron oxide | 0.27 |
| Yellow iron oxide | 0.46 |
| Titanium dioxide | 2.87 |
| Barium sulfate | 8.64 |
| Mica | 5.46 |
| Phase II | |
| Polymethyl methacrylate | 5.00 |
| polymethylsilsesquioxane | 10.00 |
| Phase III | |
| Deionized water | 22.06 |
| Isoprene glycol | 17.00 |
| Sodium hydroxide pellets | 0.33 |
| Phase IV | |
| Behenic acid | 2.81 |
| Phase V | |
| Citric acid | 0.30 |
| Phenoxyethanol/parabens | 0.70 |
| PVP/polycarbamyl polyglycol | 2.00 |
| ester | |
| Phase VI | |
| Sodium hyaluronate (2%) | 10.00 |
| Phase VII | |
| Deionized water | 8.00 |
| PPG-20 methyl glucose ether | 4.00 |

The components of Phase I are combined and mixed until uniform and then ground. The Phase II ingredients are added to Phase I and mixed until uniform. In a primary kettle, Phase III ingredients are combined and heated to 85° C. while mixing with a lightening mixer set at moderate speed. Phase IV materials are added to the batch, and mixing is continued until it is completely dissolved. While mixing, the Phase V ingredients are added in order and mixing is continued until the batch is uniform. The combined Phases I and II are then slowly added to the batch while mixing and mixing is continued until the batch is uniform. Phase VII is then added to the batch, and mixing continued until the batch is completely uniform. When the batch is uniform and deaerated, it is complete, water is added in a quantity sufficient to compensate for loss during processing, and the batch is cooled to room temperature.

Similar formulas are also prepared with PPG-20 methyl glucose distearate, methyl gluceth 10, methyl gluceth 20, glyceryl monostearate, sucrose distearate, PEG 120 methyl glucose dioleate, or cholesterol in place of PPG-20 methyl glucose ether.

What we claim is:

1. A non-emulsion topical gel composition comprising an aliphatic polyhydric alcohol, an alkali salt of a fatty acid ester, a carbohydrate-based or sterol-based surfactant/emulsifier or combination thereof, water, and substantially no oil.

2. The composition of claim 1 in which the salt is a sodium, potassium or amine salt of a C12-C22 fatty acid.

3. The composition of claim 2 in which the fatty acid is behenic acid.

4. The composition of claim 1 in which the surfactant/emulsifier is carbohydrate-based.

5. The composition of claim 4 in which the surfactant/emulsifier is sucrose- or glucose-based.

6. The composition of claim 4 in which the surfactant/emulsifier is selected from the group consisting of PPG-20 methyl glucose ether, PPG-20 methyl glucose distearate, methyl gluceth 10, methyl gluceth 20, glyceryl monostearate, sucrose distearate, or PEG 120 methyl glucose dioleate, in place of PPG-20 methyl glucose ether.

7. The composition of claim 1 in which the surfactant/emulsifier is sterol-based.

8. The composition of claim 7 in which the surfactant/emulsifier is cholesterol or a phytosterol.

9. The composition of claim 1 which contains at least one pigment.

10. The composition of claim 9 in which the pigment is selected from the group consisting of iron oxides (yellow, red, brown or black), ferric ammonium ferrocyanide(blue), manganese violet, ultramarine blue, chrome oxide(green), talc, zeolite, kaolin, titanium dioxide(white), zinc oxide, and mixtures thereof.

11. The composition of claim 9 in which the pigment is a water-dispersible pigment.

12. The composition of claim 11 in which the pigment is coated with dimethicone copolyol.

13. The composition of claim 1 in which the polyhydric alcohol comprises isoprene glycol.

14. The composition of claim 1 which is a color cosmetic.

15. A non-emulsion, topical gel composition comprising
(a) from about 10 to about 40% of an aliphatic polyhydric alcohol;
(b) from about 1 to about 15% of an amine or alkali metal salt of a fatty acid;
(c) from about 1 to about 10% of a sterol- or carbohydrate-based surfactant/emulsifier.
(d) from about 20 to about 60% of water, the composition containing substantially no oil.

16. The composition of claim 15 comprising
(a) from about 10 to about 25% of an aliphatic polyhydric alcohol;
(b) from about 1 to about 6% of an amine or alkali metal salt of a fatty acid;
(c) from about 2 to about 6% of a sterol- or carbohydrate-based surfactant
(d) from about 20 to about 60% of water, the composition containing substantially no oil.

17. The composition of claim 15 in which the salt is a sodium, potassium or amine salt of a C12-C22 fatty acid.

18. The composition of claim 15 which comprises an alkali metal salt of behenic acid.

19. The composition of claim 15 in which the polyhydric alcohol comprises isoprene glycol.

20. The composition of claim 15 in which the surfactant/emulsifier is sucrose or glucose-based.

21. The composition of claim 15 in which the surfactant/emulsifier is cholesterol or a phytosterol.

22. The composition of claim 15 in which the surfactant/emulsifier is selected from the group consisting of PPG-20 methyl glucose ether, PPG-20 methyl glucose distearate, methyl gluceth 10, methyl gluceth 20, glyceryl monostearate, sucrose distearate, or PEG 120 methyl glucose dioleate.

23. The composition of claim 15 which comprises at least one pigment.

24. The composition of claim 15, which comprises isoprene glycol, sodium behenate, a carbohydrate-based surfactant, and water.

25. The composition of claim 23 in which the pigment is a water-dispersible metal oxide.

26. The composition of claim 25 in which the pigment is coated with dimethicone copolyol.
